(19) United States
(12) Patent Application Publication
(10) Pub. No.: US 2005/0259793 A1
(43) Pub. Date: Nov. 24, 2005

(54) Medical phantom, holder and method of use thereof

(76) Inventors: In Hwan Yeo, Richmond Hill (CA); Sandra McIntosh, Athens, GA (US); Chris C.K. Wang, Chamblee, GA (US); Akbar Beiki-Ardakani, Thornhill (CA)

Correspondence Address: MYERS & KAPLAN, INTELLECTUAL PROPERTY LAW, L.L.C., 1899 POWERS FERRY ROAD, SUITE 310, ATLANTA, GA, 30339, US

(21) Appl. No.: 10/848,812

(22) Filed: May 19, 2004

(51) Int. Cl. A61N 5/10; G03B 42/04

(52) U.S. Cl. 378/182

(57) ABSTRACT

A phantom and film cassette therefor, a composition of high atomic number elements and tissue-equivalent material for a phantom, and an adjustable holder for a phantom. The film cassette comprises sections of tissue-equivalent material, wherein the sections retain a sheet of film when closed together and the phantom composition comprises tissue-equivalent material and high atomic number elements. In operation, dose distributions are determined via computation at various depths within a simulated water-equivalent phantom. After calculating dose distributions, an actual beam is delivered to a phantom containing the cassette, or utilizing a phantom containing high atomic number elements, wherein the phantom mimics human tissue, wherein the phantom houses radiographic film. Images are then generated on the film, the images are converted into an actual dose distribution, and the actual dose distribution is compared with the calculated dose distributions. Finally, a patient is treated based on the beam delivery thus verified.

### TECHNICAL FIELD

The present invention relates generally to an apparatus and method for simulating human tissue (or water) for the purpose of estimating and/or calibrating dose levels due to x-ray exposure prior to exposing a patient thereto. More specifically, the present invention relates to a composition and construction for forming a filtering apparatus to reduce x-ray film over-response to photon beams, wherein the apparatus comprises a cassette, or a water phantom, incorporating lead foil and water (or water-equivalent plastic or polymer), or alternately lead particles admixed with a water-equivalent plastic or polymer material, wherein the cassette, or a water phantom, is enclosed in a carrier for facilitating insertion into, positioning in, and removal from, x-ray equipment. The present invention is particularly advantageous in providing an easily handled cassette phantom and holder, allowing rapid and easy setup in x-ray equipment.

### BACKGROUND OF THE INVENTION

Dosimetry of radiotherapy treatment beams is rapidly becoming a very important procedure because successful radiation therapy requires an accurate delivery of a targeted dose to a cancerous volume of tissue. For example, it has been found that a dose delivery 10%-15% below the target will result in a two- to three-fold decrease in the chance of cure, while delivery of a dose higher than the target increases the chance of irreversible damage by overexposure to x-rays. Therefore, accurate and specific dose levels are critical to the success rate of patient treatment.

One such method of treating patients with x-radiation is Intensity Modulated Radiation Therapy (IMRT). With IMRT, the radiation is delivered as thousands of tiny, pencil-thin radiation beams (i.e. beamlets), wherein the beams enter the body from many angles to destroy cancer cells with accuracy. This accurate delivery of beamlets permits a higher dose of radiation to be delivered to tumors and limits the dose to surrounding healthy tissue, thereby reducing radiation side effects. In this fashion, IMRT can be utilized to safely treat tumors located near critical organs, such as the eye and spinal cord.

The positions of IMRT beams targeted to the tumor of concern are computationally optimized based on the computed tomography image of a patient. Computed tomography, or CT, is an x-ray diagnostic procedure utilized to generate a three-dimensional image of a patient, wherein the resulting image is composed of a multitude of cross-sectional views. CT requires data acquisition, image reconstruction and image display. To collect data, x-rays are passed through a patient and are attenuated within the patient, wherein the resulting levels of x-rays are sensed by external detectors to allow the creation of a detailed image of the internal composition of the patient. By moving the x-ray source and taking multiple images, detailed cross-sections can be produced, which then can be utilized to form a three-dimensional image of the patient for accurate selection of the target area to be treated.

Once the CT image has been formed and the target area position elucidated and selected, radiation therapy can be planned. In the planning, radiation beams and beamlets are optimized. Prior to performing IMRT, verification of the radiation levels for the therapy is performed. Typically, a phantom, or tissue mimic, is utilized to assist in such verification. First, beams that were optimized for patient treatment are delivered upon a flat phantom and the consequent dose distributions at some specific depth are calculated for each beam. Second, beams are actually delivered on a phantom that houses x-ray film under a medical linear accelerator, thereby generating images on the films. The images are converted into a dose distribution, which is then compared with the dose distribution obtained by calculation. If the difference between the calculations and the actual measurements are within acceptable parameters, the treatment based on the computationally optimized beam commences on a patient.

As explained in the previous paragraph, in order to achieve and/or confirm the target beam delivery, successful radiotherapy requires accurate dosimetry for treatment verification. Existing dosimeters, such as ion chambers, thermoluminescence dosimeters, and diodes, each have drawbacks including relatively long measurement time and poor spatial resolution.

An ionization chamber (IC)/water-equivalent phantom system has been recommended for isodose distribution measurement. However, ICs have some shortcomings in utilization. Measurement with an IC provides only selective information with poor spatial resolution; that is, each data point is limited by the volume of the IC and the spacing between measurements. In addition, utilization of an IC typically requires a disadvantageously long measurement time. Another method, via dynamic beam defining collimators or wedges, has complicated dose measurement using an IC. For dynamic-wedged beam dosimetry, a large array of ICs must be used to simultaneously measure doses at various positions in a phantom. In addition to an economic disadvantage, the simultaneous placement of a large number of ICs in a phantom alters the dose distribution being measured. The same disadvantages also apply to thermoluminescence dosimeters (TLDs) and diode detectors. Thus improved dosimetry methods are desirable. X-ray film may be utilized for this purpose, as it possesses the required properties discussed above as a dosimeter. However, it has heretofore been clinically questionable as a dosimeter for photon beams because, due to its silver bromide formulation, it over-responds to photons with energies below about 400 keV.

Film dosimetry, in general, can be utilized for measurement of megavoltage x-rays and thus contributes to the general quality assurance of traditional radiation therapy. The accuracy of film dosimetry for the specific verification of x-rays has been found to have an associated potential error as high as 10-20% for IMRT. Such an error level significantly exceeds the required dosimetric accuracy for radiation therapy. As will be more fully detailed hereunder, film dosimetry can be improved significantly by utilizing a cassette or a phantom that houses filters, wherein the filters prevent low-energy photons from reaching the film, thus obviating film over-response, the typical source of error. In this fashion, film dosimetry can be utilized for verification of radiation therapy.

There are various devices and methods available for creating medical phantoms, or mimics to water (or human tissue) for film dosimetry, wherein the different phantoms may be utilized for different types of analytical techniques. Each, however, is disadvantageous when compared to the present invention, as the large error associated with film dosimetry is still present.

As will be readily seen from the description below, the present invention differs from the previous use of intensifying screens based on lead or high atomic number materials in film cassettes, wherein previous uses have restricted films to placement in contact with the screens and imaging. On the contrary, the current invention provides spacing between the film and the filters with the goal of accurate radiation dosimetry (or measurement).

Other assemblies have been utilized wherein stacks of tissue-equivalent materials are placed together along with lead foil with spacing from the film. Such devices suffer from a lack of reproducibility and because they are not integral units, they are not readily handled without great care. Previously, the idea of adding lead powder in the phantom was realized. However, such realization was not rigorous in that the phantom was embrittled and thus not practically durable. In addition, the phantom was not water- or tissue-equivalent.

Therefore, it is readily apparent that there is a need for a medical phantom device and method for filtration of photons, capable of enabling the utilization of a film cassette and a phantom mimicking human tissue and containing x-ray film for radiation dose measurement, thereby avoiding the above-discussed disadvantages. There is a further need for a device to hold such a medical phantom for insertion, alignment and removal. As will be more fully detailed hereinbelow, it is to the provision of such an apparatus with holder that the present invention is directed.

### BRIEF SUMMARY OF THE INVENTION

Briefly described, in a preferred embodiment, the present invention overcomes the above-mentioned disadvantages and meets the recognized need for such a device and method by providing embodiments directed to a medical phantom cassette for use with x-ray film, wherein the cassette includes lead, preferably in the form of foil, predisposed within the cassette body. The cassette is installed within a holding device that can readily be inserted into medical equipment, aligned therewithin, and removed upon completion of verification of the correct dose for patient treatment

According to its major aspects and broadly stated, the present invention, in its preferred form, is a medical phantom cassette and method for mimicking human tissue for the purpose of verification of medical apparatus, particularly x-ray equipment for IMRT, such as a medical linear accelerator and radiation therapy. The cassette includes two sections, each having a water-equivalent material construction, wherein the sections retain a piece of x-ray film when closed together. The cassette is retained within a holding cartridge that enables compression of the medical phantom and x-ray film, wherein the holding cartridge has legs for height adjustment for adaptable setup within an x-ray machine.

More specifically, the present invention is a composition of materials that filters x-ray photons and that can be formed into a suitable prismatic film dosimetry cassette. The two-section cassette has filters located within the body of each section, wherein the top, bottom and side surfaces of the sections form a generally rectangular-shaped prism. The bodies of the sections are fabricated from tissue-equivalent plastic or polymeric materials, such as, for exemplary purposes only, polystyrene or water-equivalent plastic, which serves to mimic human tissue when bombarded by photons. The sections are hingably attached to facilitate opening and closing, and the filters are made of a high atomic weight element sheet material.

In use, film is inserted between the sections and the cassette is closed with the film retained therein. The film-containing cassette can then be augmented with additional slabs of tissue mimicking material, such as, for exemplary purposes only, a sandwich of polystyrene slabs or slabs of water-equivalent material. Following such augmentation, the cassette is placed within a holder for positioning within an x-ray machine, wherein the holder facilitates compression and height positioning adjusting setup parameters such as alignment and flatness.

The present invention also includes the idea of a water-equivalent phantom containing the elements of filtering materials, such as lead or high atomic number materials, uniformly distributed in a phantom body. Film can be sandwiched in between the slabs of the phantom composed of the mixture of plastic materials and high atomic number elements. The size and shape of the phantom can be determined depending on the application (simulation of a rectangular water phantom or any human organ).

A feature and advantage of the present invention is its ability to mimic human tissue in any desired shape to enable accurate measurement of x-ray dose for radiation therapy verification such as IMRT, and additionally the calibration of x-ray equipment.

A feature and advantage of the present invention is its ability to accurately verify the planned dose delivery to a patient and help prevent over- and under-exposure of a patient to radiation.

A further feature and advantage of the present invention is that it facilitates easy insertion into, and removal from, x-ray equipment.

An additional feature and advantage of the present invention is its ability to be adjusted while positioned within medical equipment.

A further feature and advantage of the present invention is its ease of use, manufacture and low cost of production.

These and other features and advantages of the present invention will become more apparent to one skilled in the art from the following description and claims when read in light of the accompanying drawings.

### BRIEF DESCRIPTION OF THE DRAWINGS

Having thus described the invention in general terms, the present invention will be better understood by reading the Detailed Description of the Preferred and Selected Alternate Embodiments with reference to the accompanying drawing figures, which are not necessarily drawn to scale, and in which like reference numerals denote similar structures and refer to like elements throughout, and in which:

FIG. 1A depicts a film dosimetry cassette according to a preferred embodiment of the present invention;

FIG. 1B depicts the film dosimetry cassette of FIG. 1A with a sheet of film retained therein;

FIG. 2 depicts the film dosimetry cassette device of FIG. 1B showing an augmented phantom set;

FIG. 3A depicts a device for holding the construction of FIG. 1A according to a preferred embodiment of the present invention;

FIG. 3B depicts the device of FIG. 2 within the holding device of FIG. 3A according to a preferred embodiment of the present invention; and

FIG. 4 depicts an alternate embodiment of the present invention.

### DETAILED DESCRIPTION OF THE PREFERRED AND SELECTED ALTERNATE EMBODIMENTS

In describing the preferred and selected alternate embodiments of the present invention, as illustrated in the Figures, specific terminology is employed for the sake of clarity. The invention, however, is not intended to be limited to the specific terminology so selected, and it is to be understood that each specific element includes all technical equivalents that operate in a similar manner to accomplish similar functions.

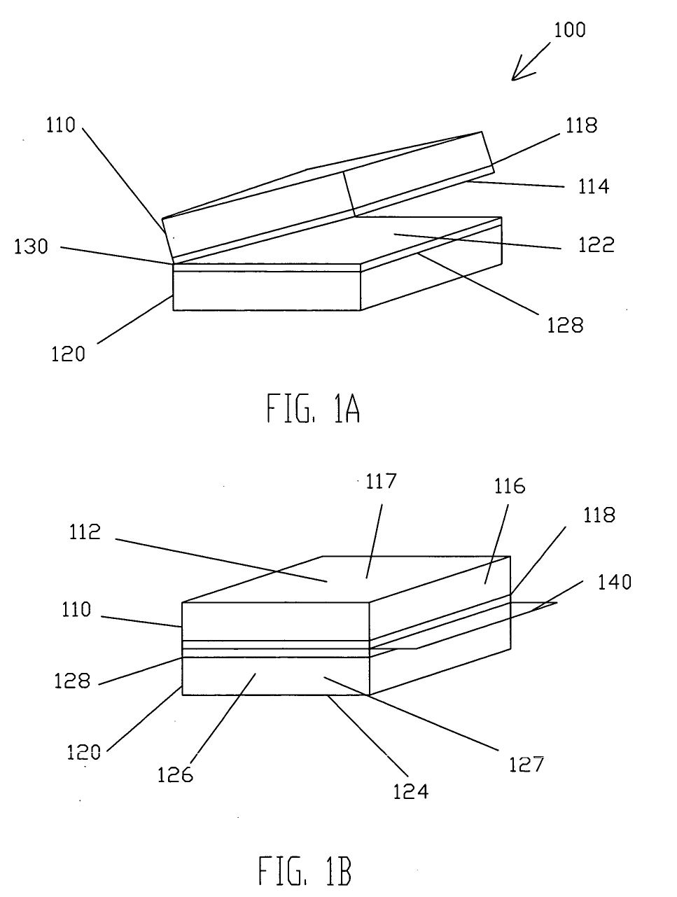

Referring now to FIGS. 1A and 1B and the preferred form of the present invention, cassette 100 is preferably a prismatic film dosimetry cassette comprising first section 110 and second section 120. First section 110 preferably comprises top surface 112, bottom surface 114, side surfaces 116, body 117 and filter 118, and second section 120 preferably comprises top surface 122, bottom surface 124, side surfaces 126, body 127 and filter 128. Top surfaces 112 and 122, bottom surfaces 114 and 124 and side surfaces 116 and 126 of first and second sections 110 and 120, respectively, preferably form a generally rectangular-shaped prism, wherein bodies 117 and 127 are preferably fabricated from a plastic or polymeric material, such as, for exemplary purposes only, polystyrene plastic or other water- or tissue-equivalent material. First section 110 and second section 120 are preferably integral units and are hingably attached via hinge 130 in order to preferably facilitate alignment and closure. Filters 118 and 128 preferably comprise a sheet of high atomic weight element material, such as, for exemplary purposes only, lead foil, wherein filters 118 and 128 are preferably integral to first section 110 and section 120, respectively.

First section 110 and second section 120 are preferably comprised of bodies 117 and 127, respectively, wherein bodies 117 and 127 preferably serve to mimic human tissue when bombarded by photons. Filters 118 and 128 are preferably located within first section 110 and second section 120, respectively, preferably within bodies 117 and 127, respectively. Filter 118 is preferably carried at a distance of approximately 0.6 cm from bottom 114 of first section 110 and filter 128 is preferably carried at approximately 0.6 cm from top 122 of second section 120. Although such positioning of filters 118 and 128 is preferred, other positions could be utilized, either closer or farther from the top or bottom of either or both sections.

The hinged relationship of first section 110 and second section 120 of cassette 100 preferably enables the placement and fixable retention of film 140 therewithin.

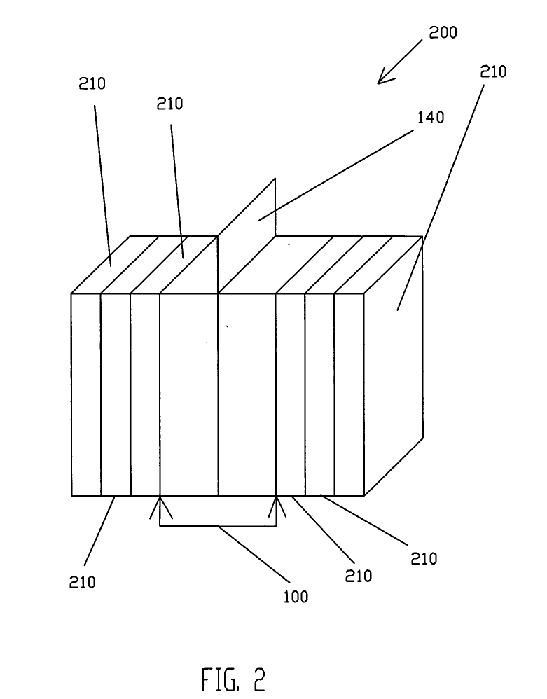

Referring now to FIG. 2, cassette 100 containing film 140 is preferably positioned within slabs 210 comprising augmented phantom 200, wherein slabs 210 are preferably formed from tissue-mimicking material, such as, for exemplary purposes only, a sandwich of polystyrene slabs or other water- or tissue-equivalent material. At least one slab 210 is preferably positioned proximate top surface 112 of first section 110 and at least one slab 210 is preferably positioned proximate bottom surface 124 of second section 120, wherein slabs 210 and cassette 100 preferably form a sandwich construction.

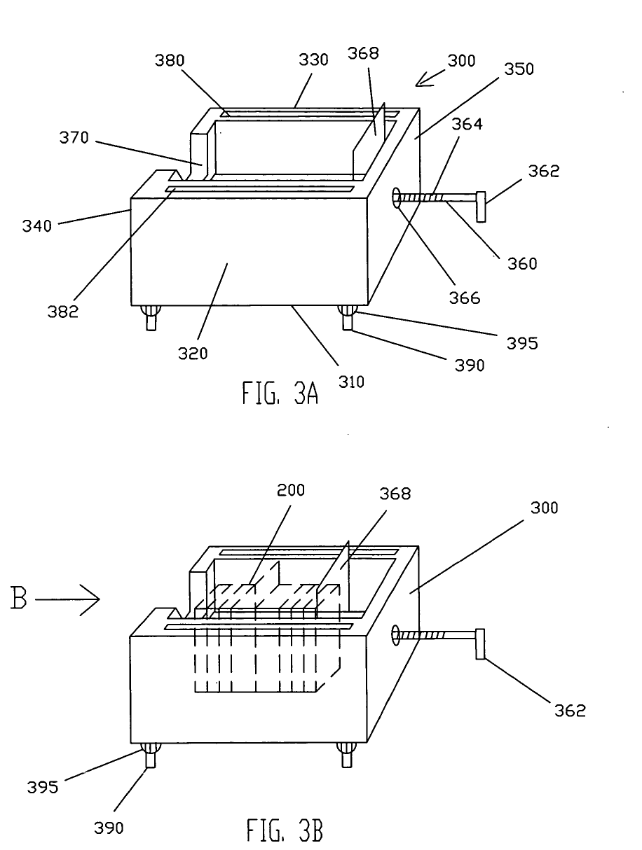

Referring now to FIG. 3A, holder 300 is preferably defined by base 310, side wall 320, side wall 330, front wall 340 and back wall 350, and holder 300 is preferably formed from clear plastic materials. Compression device 360, preferably located within back wall 350, preferably has handle 362, threaded shaft 364, threaded bushing 366 and compression plate 368. Threaded bushing 366 is preferably retained within back wall 350.

Referring now to FIG. 3B, the cassette 100/phantom 200 combination as shown in FIG. 2 is preferably positioned in holder 300 and preferably compressed via application of force preferably by turning handle 362 of compression device 360. Front wall 340 preferably has opening 370 defined therein to preferably permit entry of photon beam B. Rulers 380 and 382 are preferably located atop side walls 330 and 320, respectively, to preferably enable measurement of the extent of compression and reproducibility of a selected cassette 100/phantom 200 arrangement. Legs 390 are preferably attached to base 310, wherein legs 390 preferably enable height adjustment via mechanism 395, wherein mechanism 395 is, for exemplary purposes only, a screw adjustment.

In use, calculated values are preferably obtained for a beam delivered on simulated water-equivalent phantom 200 located perpendicular to, or parallel with, beam B. Holder 300, with cassette 100 therein, is then preferably positioned within an x-ray machine, such as, for exemplary purposes only, a medical linear accelerator. Mechanism 395 is preferably subsequently utilized to adjust the height of holder 300 within the x-ray machine, thereby preferably facilitating accurate actual dosage measurement by film exposure to radiation. In this fashion, x-ray film is preferably exposed to a known radiation dose, and correlation with the calculated dosage values is preferably verified by utilizing the exposure determined from the film. Once verified, human exposure at known quantified levels preferably takes place.

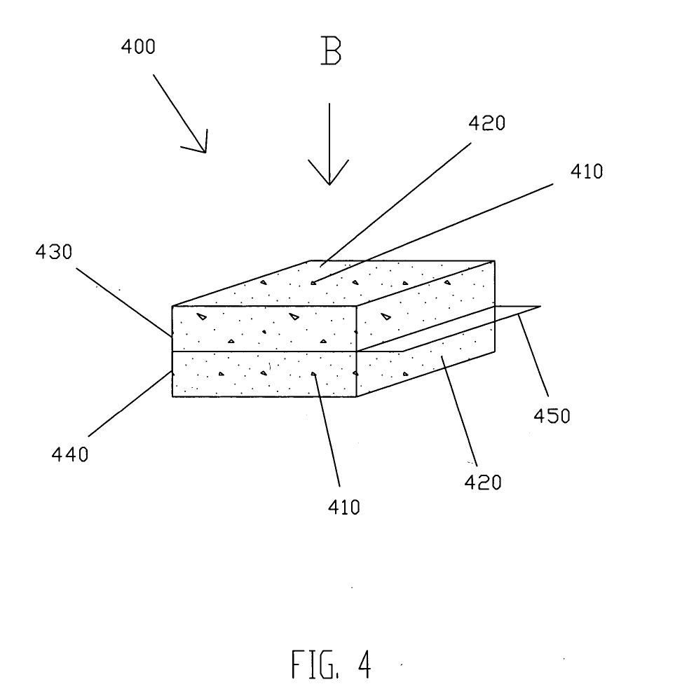

It is envisioned in an alternate embodiment represented by FIG. 4 that phantom 400 of the present invention could be formed from a composition of high atomic number powder 410 and plastic or polymeric compound 420, wherein high atomic number powder 410 comprises, for exemplary purposes only, lead and/or tungsten powder, or other Group VI element from the periodic table, thereby making the entire phantom tissue-equivalent. The high atomic number powder could comprise, for example, approximately between 5 to 6 percent by weight if lead or tungsten is utilized. Other high atomic number powders, and combinations thereof, could be utilized for this embodiment of the present invention, and the percentage composition could require variation for use with powders other than lead and/or tungsten. The preferred, but not limiting to, elemental composition in weight percent is approximately 80.5% for carbon (C), approximately 13.5% for hydrogen (H), and approximately 6.0% for tungsten (W).

In this embodiment, high atomic number powder 410 and plastic or polymer 420 could be mixed together to form phantom slabs 430 and 440, wherein phantom slabs 430 and 440 comprise the entire phantom and could be placed on opposing sides of x-ray film 450, in order to prevent overresponse of the x-ray film. Phantom slabs 430 and 440 thereby form phantom 400, wherein phantom 400 is suitable for mimicking human tissue in response to a photon beam B without requiring augmentation. Phantom 400 could then be utilized for verification of the intensity of radiation beams for patient treatment as is further described hereinbelow.

In operation of the various embodiments of the present invention hereinabove, the intensity of radiation beams intended for patient treatment can be verified by 1) obtaining a cassette or a phantom of the present invention for mimicking human tissue; 2) computationally delivering the radiation beams intended for patient treatment on the surface of a simulated tissue- or water-equivalent phantom; 3) calculating the dose distributions at a specific depth below the surface of the phantom for each beam component; 4) setting up radiation beams for actual delivery on radiographic film, using either a cassette in an augmented phantom or stand-alone phantom of the present invention to house the film; 5) delivering an actual radiation beam intended for patient treatment onto the augmented or the phantom of the present invention, whereby images are generated on the film; 6) converting the images into equivalent actual dose distributions; 7) comparing the actual dose distributions with the calculated dose distributions; and 8) determining whether the differences between the calculated values and the values from actual images are within acceptable levels. Finally, a patient is preferably treated using the verified beams.

It is envisioned in an alternate embodiment that a high atomic number powder could be mixed with a tissue- or water-equivalent plastic or polymer and formed into a humanoid shape.

The foregoing description and drawings comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely numbering or listing the steps of a method in a certain order does not constitute any limitation on the order of the steps of that method. Many modifications and other embodiments of the invention will come to mind to one skilled in the art to which this invention pertains having the benefit of the teachings presented in the foregoing descriptions and the associated drawings. Although specific terms may be employed herein, they are used in a generic and descriptive sense only and not for purposes of limitation. Accordingly, the present invention is not limited to the specific embodiments illustrated herein, but is limited only by the following claims.

1. A film cassette comprising:
a first section and a second section, wherein said first section and said second section comprise tissue-equivalent material, and wherein said cassette further comprises at least one lead foil sheet carried within said first section and at least one lead foil sheet carried within said second section.

2. The film cassette of claim 1, wherein said first section and said second section are generally prismaticly shaped.

3. The film cassette of claim 1, wherein said first section and said second section are fabricated of a plastic material.

4. The film cassette of claim 3, wherein said plastic material is a water-equivalent plastic.

5. The film cassette of claim 1, wherein said first section and said second section can retain a sheet of film therebetween.

6. The film cassette of claim 5, wherein said lead foil sheets are carried within said first and second sections at a distance of approximately 6 millimeters from the position of the sheet of film as retained therein.

7. The film cassette of claim 1, wherein said first section and said second section are hingably related.

8. A medical phantom comprising:
a film cassette comprising a first section and a second section, wherein said first section and said second section comprise tissue-equivalent material, and wherein said cassette further comprises at least one lead foil sheet carried within said first section and at least one lead foil sheet carried within said second section, and wherein said first section and said second section have outer surface sides, film;
at least one slab of tissue-equivalent material positioned proximate said outer surface side of said first section; and
and at least one slab of tissue-equivalent material positioned proximate said outer surface side of said second section.

9. The medical phantom of claim 8, wherein said slabs comprise water-equivalent plastic.

10. A holder for a medical phantom comprising:
a container, wherein said container comprises bottom, first side, second side, front and back, wherein said bottom, said first side, said second side, said front and said back comprise a clear plastic material.

11. The holder for a medical phantom of claim 10, further comprising legs.

12. The holder for a medical phantom of claim 11, wherein said legs further comprise means for adjusting the length thereof.

13. A medical phantom comprising:
at least one high atomic number powder; and
a tissue-equivalent plastic compound.

14. The medical phantom of claim 13, wherein said at least one high atomic number powder comprises at least one element selected from Group VI of The Periodic Table of the Elements.

15. The medical phantom of claim 13, wherein said at least one high atomic number powder comprises at least one element selected from the group consisting of lead and tungsten.

16. The medical phantom of claim 13, having a concentration of said at least one high atomic number powder comprising approximately 6% by weight.

17. The medical phantom of claim 13, having a concentration of said tissue-equivalent plastic compound comprising approximately 94% by weight.

18. The medical phantom of claim 13, comprising approximately 80.5% carbon by weight and approximately 13.5% hydrogen by weight.

19. A method of verifying intensity of radiation beams intended for patient treatment, wherein said radiation beams comprise beam components, said method comprising the steps of:
a. obtaining a phantom for mimicking human tissue, wherein said phantom has a generally flat surface, and wherein said phantom comprises a film cassette comprising a first section and a second section, wherein said first section and said second section comprise tissue-equivalent material, and wherein said cassette further comprises at least one lead foil sheet carried within said first section and at least one lead foil sheet carried within said second section, and wherein said first section and said second section have outer surface sides; film; at least one slab of tissue-equivalent material positioned proximate said outer surface side of said first section; and at least one slab of tissue-equivalent material positioned proximate said outer surface side of said second section;
b. computationally delivering said radiation beams intended for patient treatment on said phantom;
c. calculating dose distributions at a specific depth below the surface of said phantom for each of said beam components;
d. setting up radiation beams for actual delivery on said phantom, wherein said phantom houses radiographic film.
e. delivering actual radiation beams intended for patient treatment on said flat phantom, whereby images are generated on the film;
f. converting said images into equivalent actual dose distributions; and
g. comparing said actual dose distributions with said calculated dose distributions.

20. The method of claim 19, wherein said phantom is contained within a cassette.

21. The method of claim 19, further comprising the step of:
determining whether the differences between said actual dose distributions and said calculated dose distributions are within acceptable levels.

22. The method of claim 19, further comprising the step of:
treating a patient using a verified beam.

23. The method of claim 22, wherein said verified beam is delivered via a medical linear accelerator.

24. A method of verifying intensity of radiation beams intended for patient treatment, wherein said radiation beams comprise beam components, said method comprising the steps of:
a. obtaining a phantom for mimicking human tissue, wherein said phantom has a generally flat surface, and wherein said phantom comprises at least one high atomic number powder and at least one tissue-equivalent plastic compound;
b. computationally delivering said radiation beams intended for patient treatment on said phantom;
c. calculating dose distributions at a specific depth below the surface of said phantom for each of said beam components;
d. setting up radiation beams for actual delivery on said phantom, wherein said phantom houses radiographic film.
e. delivering actual radiation beams intended for patient treatment on said flat phantom, whereby images are generated on the film;
f. converting said images into equivalent actual dose distributions; and
g. comparing said actual dose distributions with said calculated dose distributions.

25. A method of exposing film in an x-ray machine comprising the steps of:
a. inserting film between layers of tissue-mimicking material to form a sandwich;
b. placing said sandwich in a holding device comprising a chamber, a compression mechanism and legs, wherein said legs have a height adjusting mechanism;
c. inserting said holding device into an x-ray machine;
d. adjusting the height of said holding device via said height adjusting mechanism; and
e. exposing said film to radiation from the x-ray machine.
